(19) United States
(12) Patent Application Publication
(10) Pub. No.: US 2024/0376047 A1
(43) Pub. Date: Nov. 14, 2024

(54) COLLECTOR, FLOTATION METHOD, AND COMPOUND

(71) Applicants: Akita University, Akita (JP); SUMITOMO CHEMICAL COMPANY, LIMITED, Tokyo (JP)

(72) Inventors: Atsushi SHIBAYAMA, Akita (JP); Manabu YAMADA, Akita (JP); Kazutoshi HAGA, Akita (JP); Jia ZHAO, Akita (JP); Ayumi ASANO, Osaka (JP)

(21) Appl. No.: 18/691,225

(22) PCT Filed: Jul. 4, 2022
(86) PCT No.: PCT/JP2022/026599

§ 371 Date: Mar. 12, 2024

(30) Foreign Application Priority Data
Sep. 14, 2021 (JP) 2021-149785
Feb. 25, 2022 (JP) 2022-027875

(51) Int. Cl. C07C 321/28; B03D 1/012; B03D 1/02

(52) U.S. Cl. CPC C07C 321/28; B03D 1/012; B03D 1/02; B03D 2201/02; B03D 2203/02

(57) ABSTRACT

Provided are: A collector containing a compound that increases the recovery amount of a target mineral; a flotation method that uses this compound; and a compound that is suitable as a collector. This collector includes a compound represented by the following formula (1).

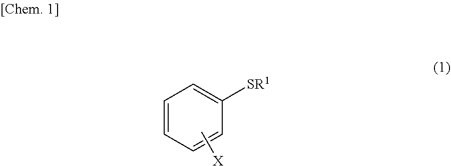

(In formula (1), R¹ represents an alkyl group having 1 or more and 18 or less carbon atoms, and X represents NR²R³, SR⁴, or OR⁵, and R²⁻³ each represents hydrogen or an alkyl group having 1 or more and 18 or less carbon atoms, and R⁴⁻⁵ each represent an alkyl group having 1 or more and 18 or less carbon atoms.

### TECHNICAL FIELD

The present disclosure relates to a collector, a flotation method, and a compound.

### BACKGROUND ART

Flotation is conventionally known to recover minerals contained in ore.

For example, Patent Document 1 discloses a method of recovering copper using dodecyl mercaptan and 2-mercaptobenzothiazole as collectors used in flotation.

However, with the copper recovery method using dodecyl mercaptan and 2-mercaptobenzothiazole disclosed in Patent Document 1, the recovered amount of copper is insufficient.

Therefore, a collector that increases the recovery amount of a target mineral has been awaited. Furthermore, the development of compounds useful in collectors has been advanced.

### CITATION LIST

### Patent Document

Patent Document 1: Japanese Unexamined Patent Application, Publication No. 2006-307293

### DISCLOSURE OF THE INVENTION

### Problems to be Solved by the Invention

It is an object of the present disclosure to provide a collector including a compound that increases the recovery amount of a target mineral, a flotation method using the compound, and a compound suitable for a collector.

### Means for Solving the Problems

[1] A collector including a compound represented by the following formula (1):

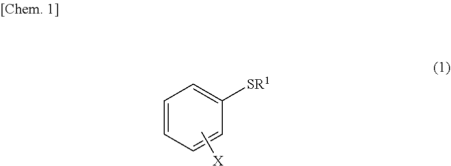

where R¹ represents an alkyl group having 1 or more and 18 or less carbon atoms, and X represents NR²R³, SR⁴, or OR⁵, and R²⁻³ each represent hydrogen or an alkyl group having 1 or more and 18 or less carbon atoms, and R⁴⁻⁵ each represent an alkyl group having 1 or more and 18 or less carbon atoms.

[2] In the collector according to [1] above, R¹ represents an alkyl group having 5 or more and 10 or less carbon atoms.

[3] In the collector according to [1] or [2] above, X represents NR²R³ or SR⁴.

[4] In the collector according to any one of [1] to [3] above, R² and R³ are each hydrogen.

[5] In the collector according to any one of [1] to [3] above, R⁴ represents an alkyl group having 5 or more and 10 or less carbon atoms.

[6] In the collector according to [1] or [2] above, R⁵ represents an alkyl group having 5 or more and 10 or less carbon atoms.

[7] In the collector according to any one of [1] to [6] above, the collector is a collector for flotation of one or more kinds of minerals each including one or more kinds of metals selected from the group consisting of Cu, Au, Zn, Pb, Pt, Pd, Rh, Ni, and Co.

[8] A flotation method comprising: adding a compound represented by the following formula (1) and a frother to an ore slurry to float one or more kinds of minerals in the ore slurry and recovering the one or more kinds of minerals:

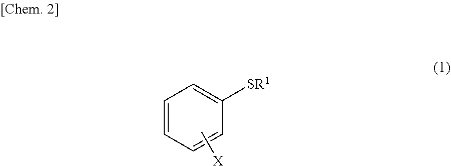

where R¹ represents an alkyl group having 1 or more and 18 or less carbon atoms, and X represents NR²R³, SR⁴, or OR⁵, and R²⁻³ each represent hydrogen or an alkyl group having 1 or more and 18 or less carbon atoms, and R⁴⁻⁵ each represent an alkyl group having 1 or more and 18 or less carbon atoms.

[9] In the flotation method according to [8] above, the ore slurry is a slurry of ore containing one or more kinds of minerals each including one or more kinds of metals selected from the group consisting of Cu, Au, Zn, Pb, Pt, Pd, Rh, Ni, and Co.

[10] In the flotation method according to [8] or [9] above, an addition amount of the compound represented by the formula (1) is 0.1 g or more and 1000 g or less per 1000 kg of ore.

[11] In the flotation method according to any one of [8] to [10] above, the ore slurry has a pH of 6 or more and 12 or less.

[12] A compound represented by the following formula (3-1-1):

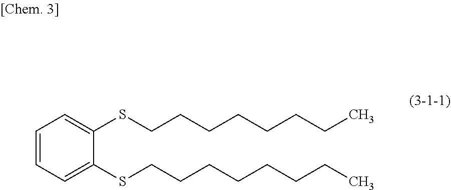 [SMILES: CCCCCCCCSc1ccccc1SCCCCCCCC]

### Effects of the Invention

According to the present disclosure, it is possible to provide a collector including a compound that increases the recovery amount of a target mineral, a flotation method using the compound, and a compound suitable for a collector.

### BRIEF DESCRIPTION OF THE DRAWINGS

FIG. 1 is an X-ray diffraction pattern of the chalcopyrite used in Examples 1-1 to 1-6 and Comparative Example 1-1.

FIG. 2 is an X-ray diffraction pattern of the ore used in Example 4-1 and Comparative Example 4-1.

FIG. 3 shows the content ratio of metals contained in the ore used in Example 4-1 and Comparative Example 4-1.

FIG. 4 is an X-ray diffraction pattern of the copper sulfide ore used in Examples 5-1 to 5-8 and Comparative Examples 5-1 and 5-2.

### PREFERRED MODE FOR CARRYING OUT THE INVENTION

Hereinafter, the present invention will be described in detail based on an embodiment.

As a result of extensive research, the present inventors have found a compound that improves the recovery amount of a target mineral. Furthermore, the present inventors have found that a collector including the above compound increases the recovery amount of the target mineral compared to conventional collectors. The present disclosure has been completed based on such findings.

First, a collector according to an embodiment of the present disclosure will be described.

The collector of the present embodiment includes a compound represented by the following formula (1).

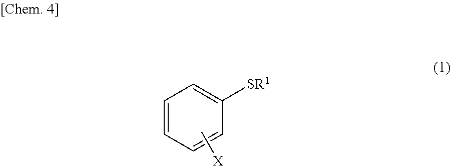

In the above formula (1), R¹ represents an alkyl group having 1 or more and 18 or less carbon atoms, and X represents NR²R³, SR⁴, or OR⁵. R²⁻³ each represent hydrogen or an alkyl group having 1 or more and 18 or less carbon atoms, and R⁴⁻⁵ each represent an alkyl group having 1 or more and 18 or less carbon atoms.

From the viewpoint of improving the recovery amount of a mineral, in formula (1), R¹ represents an alkyl group having 1 or more and 18 or less carbon atoms, and preferably an alkyl group having 5 or more and 10 or less carbon atoms.

The compound in which X in the formula (1) is NR²R³ is represented by the following formula (2).

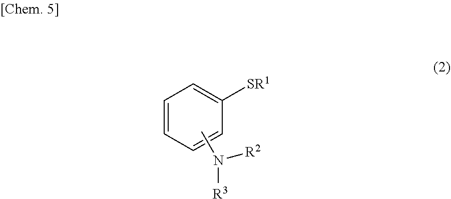

In the compound represented by the formula (2), the amine that binds to the benzene ring is introduced for the purpose of enhancing electron donating property. Furthermore, sulfur that binds to the benzene ring is introduced.

From the viewpoint of improving the recovery amount of a mineral, in the formula (2), R² and R³ are preferably each hydrogen. The compound in which R² and R³ in the formula (2) are each hydrogen is represented by the following formula (2-1).

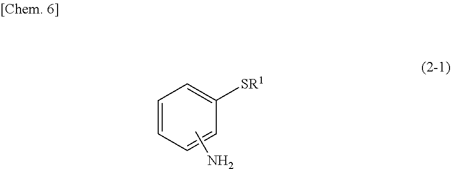

From the viewpoint of improving the recovery amount of a mineral, in the formula (2) and the formula (2-1), R¹ is preferably an alkyl group having 1 or more and 18 or less carbon atoms, and is more preferably an alkyl group having 5 or more and 10 or less carbon atoms.

Among them, from the viewpoints of imparting hydrophobicity, solubility in water, solubility in a diluent such as an organic solvent, stability, and the like, in the formula (2), R¹ is preferably an alkyl group having 8 carbon atoms (octyl group), and R² and R³ are preferably each hydrogen. This compound is represented by the following formula (2-2).

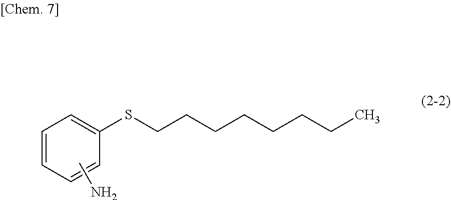

The compound represented by the formula (2-2) includes at least one kind of compound of the compound represented by the formula (2-2-1), the compound represented by the formula (2-2-2), and the compound represented by the formula (2-2-3), which are isomers.

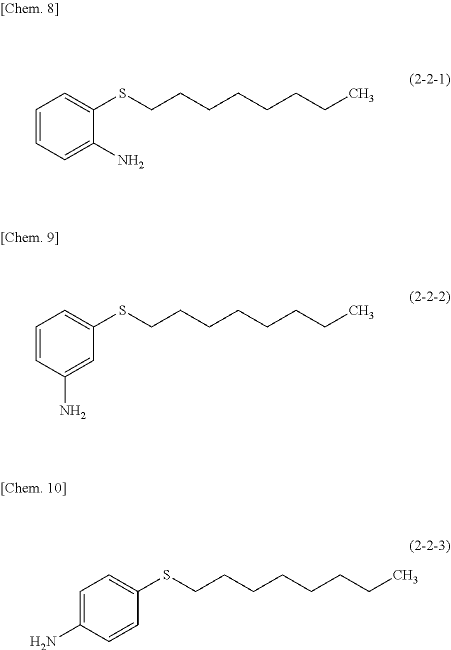 [SMILES: CCCCCCCCSc1ccccc1N ; CCCCCCCCSc1cccc(N)c1 ; CCCCCCCCSc1ccc(N)cc1]

The compound represented by the formula (2) can be synthesized by a reaction represented by the following reaction formula.

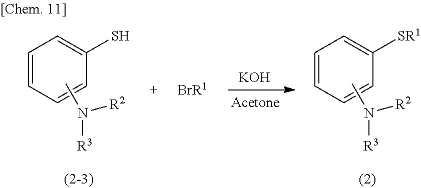

First, under a nitrogen gas flow, a compound represented by the formula (2-3), alkyl bromide, and potassium hydroxide are heated and refluxed while stirring in acetone. Subsequently, the obtained sample is washed, dehydrated, and dried. Thus, the compound represented by the formula (2) can be obtained. When the compound represented by the formula (2-1) is obtained, aminobenzenethiol is used as the compound represented by the formula (2-3). When synthesizing an ortho-substituted form of the compound represented by the formula (2), such as the compound represented by the formula (2-2-1), the ortho form of the compound represented by the formula (2-3) is used. When synthesizing a meta-substituted form of the compound represented by the formula (2), such as the compound represented by the formula (2-2-2), the meta form of the compound represented by the formula (2-3) is used. When synthesizing a para-substituted form of the compound represented by the formula (2), such as the compound represented by the formula (2-2-3), the para form of the compound represented by the formula (2-3) is used.

The compound in which X is SR⁴ in the formula (1) is represented by the following formula (3).

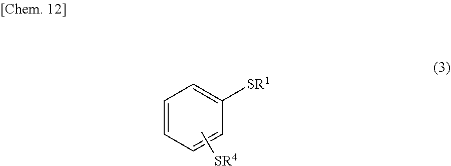

In the compound represented by the formula (3), two sulfur atoms that bind to the benzene ring are introduced.

From the viewpoint of improving the recovery amount of a mineral, in the formula (3), R¹ is preferably an alkyl group having 1 or more and 18 or less carbon atoms, and is more preferably an alkyl group having 5 or more and 10 or less carbon atoms.

From the viewpoint of improving the recovery amount of a mineral, in the formula (3), R⁴ represents an alkyl group having 1 or more and 18 or less carbon atoms, and preferably an alkyl group having 5 or more and 10 or less carbon atoms.

Among them, from the viewpoints of imparting hydrophobicity, solubility in water, solubility in a diluent such as an organic solvent, stability, and the like, in the formula (3), R¹ and R⁴ are preferably each an alkyl group having 8 carbon atoms (octyl group). The compound in which R¹ and R⁴ are each an alkyl group having 8 carbon atoms is represented by the following formula (3-1).

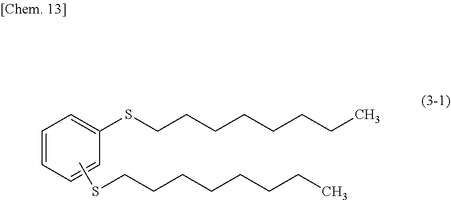

The compound represented by the formula (3-1) includes at least one kind of compound of the compound represented by the formula (3-1-1), the compound represented by the formula (3-1-2), and the compound represented by the formula (3-1-3), which are isomers.

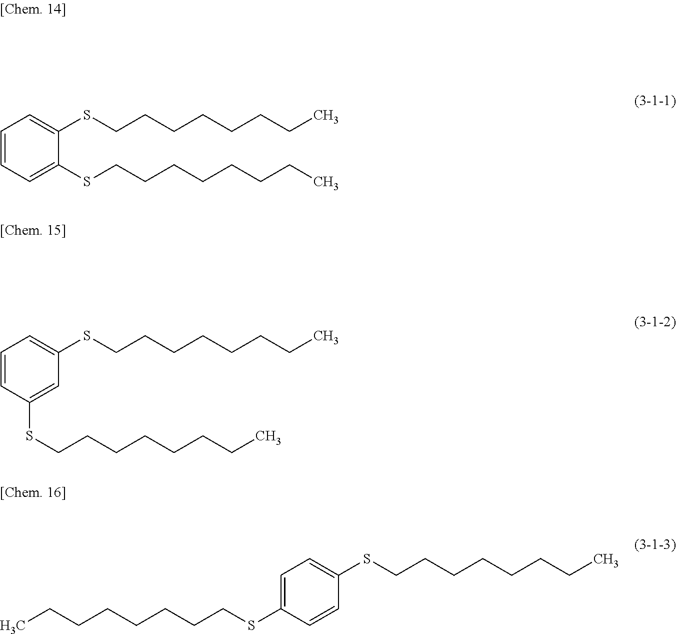 [SMILES: CCCCCCCCSc1ccccc1SCCCCCCCC ; CCCCCCCCSc1cccc(SCCCCCCCC)c1 ; CCCCCCCCSc1ccc(SCCCCCCCC)cc1]

The compound represented by the formula (3) can be synthesized by a reaction represented by the following reaction formula.

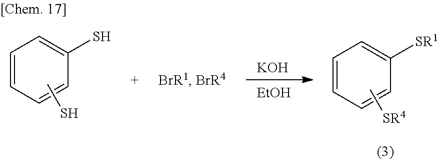

First, under a nitrogen gas flow, benzenedithiol, one or more kinds of alkyl bromides (for example, BrR¹ and BrR⁴ in the above formula), and potassium hydroxide are heated and refluxed while stirring in ethanol. Subsequently, the obtained sample is washed, dehydrated, and dried. Thus, the compound represented by the formula (3) can be obtained. When synthesizing an ortho-substituted form of the compound represented by the formula (3), such as the compound represented by the formula (3-1-1), 1,2-benzenedithiol is used. When synthesizing a meta-substituted form of the compound represented by the formula (3), such as the compound represented by the formula (3-1-2), 1,3-benzenedithiol is used. When synthesizing a para-substituted form of the compound represented by the formula (3), such as the compound represented by the formula (3-1-3), 1,4-benzenedithiol is used.

The compound in which X is OR⁵ in the formula (1) is represented by the following formula (4).

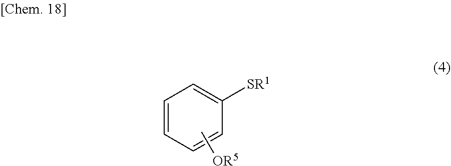

In the compound represented by the formula (4), sulfur and oxygen that bind to the benzene ring are introduced.

From the viewpoint of improving the recovery amount of a mineral, in the formula (4), R¹ is preferably an alkyl group having 1 or more and 18 or less carbon atoms, and is more preferably an alkyl group having 5 or more and 10 or less carbon atoms.

From the viewpoint of improving the recovery amount of a mineral, in the formula (4), R⁵ represents an alkyl group having 1 or more and 18 or less carbon atoms, and preferably an alkyl group having 5 or more and 10 or less carbon atoms.

The compound represented by the formula (4) can be synthesized by a reaction represented by the following reaction formula.

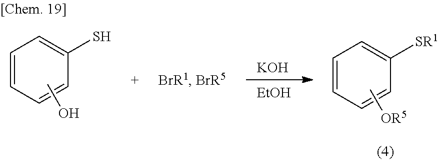

First, under a nitrogen gas flow, mercaptophenol, one or more kinds of alkyl bromides (for example, BrR¹ and BrR⁵ in the above formula), and potassium hydroxide are heated and refluxed while stirring in ethanol. Subsequently, the obtained sample is washed, dehydrated, and dried. Thus, the compound represented by the formula (4) can be obtained. When synthesizing an ortho-substituted form of the compound represented by the formula (4), 2-mercaptophenol is used. When synthesizing a meta-substituted form of the compound represented by the formula (4), 3-mercaptophenol is used. When synthesizing a para-substituted form of the compound represented by the formula (4), 4-mercaptophenol is used.

From the viewpoint of improving the recovery amount of the target mineral, among the compounds represented by the above formula (1), X is preferably NR²R³ as in the compound represented by the formula (2), or X is preferably SR⁴ as in the compound represented by the formula (3). Among the compounds in which X is NR²R³, R² and R³ are preferably each hydrogen as in the compound represented by the formula (2-1). The target mineral is one or more kinds of minerals each including one or more kinds of metals selected from the group consisting of Cu, Au, Zn, Pb, Pt, Pd, Rh, Ni, and Co.

The compound represented by the above formula (1) can increase the recovery amount of the target mineral, and in particular, can increase the recovery amount of a plurality of kinds of minerals including a plurality of kinds of metals, compared to conventional collectors. Therefore, the collector including the compound represented by the formula (1) can improve the recovery amount of a mineral, in particular, the recovery amount of a plurality of kinds of minerals including a plurality of kinds of metals. Such a collector of the present embodiment is suitably used for flotation.

The collector including the compound represented by the above formula (1) is preferably a collector for flotation of one or more kinds of minerals each including one or more kinds of metals selected from the group consisting of Cu, Au, Zn, Pb, Pt, Pd, Rh, Ni, and Co, and more preferably a collector for flotation of one or more kinds of minerals each including one or more kinds of metals selected from the group consisting of Cu, Au, Zn, and Pb. In flotation, the compound represented by the formula (1) can particularly improve the recovery amount of one or more kinds of minerals each including the above one or more kinds of metals. Therefore, when the collector including the compound represented by the formula (1) is used as the above collector for flotation, the recovery amount of one or more kinds of minerals can be increased.

The form of the collector of the present embodiment can be selected as appropriate according to the process of flotation. For example, when only a collector is added to an ore slurry, the collector of the present embodiment includes a frother in addition to the compound represented by the formula (1). When a collector and a frother are added to an ore slurry, the collector of the present embodiment may not include a frother. As the frother, a frother used in conventional flotation can be used. Furthermore, if necessary, the collector of the present embodiment may contain various additives such as an inhibitor.

Next, a flotation method according to an embodiment of the present disclosure will be described.

In the flotation method of the present embodiment, a compound represented by the above formula (1) and a frother are added to an ore slurry to float one or more kinds of minerals in the ore slurry, and the one or more kinds of minerals are recovered. In the flotation method, in addition to the compound represented by the above formula (1) and the frother, various additives such as an inhibitor may be added to the ore slurry.

In the flotation method, the compound represented by the formula (1) and the frother are added to the ore slurry to float the one or more kinds of minerals collected by the compound represented by the formula (1) together with froth generated by the frother on the liquid surface of the ore slurry. Metals can be recovered from the ore slurry by recovering such a froth layer including the minerals from the ore slurry.

The ore slurry used in the flotation method is obtained by mixing pulverized ore obtained by pulverizing ore including the desired minerals, with a liquid such as water. The frother is a substance that dissolves in a solvent to stabilize froth in a solution. Specific examples of the frother include, but are not limited to, methylisobutylcarbinol (MIBC), pine oil, and Aero froth 70 (CYTEC). The amount of the frother is preferably 0.001 g/t or more and 2000 g/t or less (0.001 g or more and 2000 g or less per 1000 kg of the ore). When the amount of the frother is 0.001 g/t or more, float ore is easily obtained, and when the amount of the frother is more than 2000 g/t, the effect of the addition of the frother may reach a plateau. In the flotation method, the ore slurry to which the compound represented by the formula (1) and the frother are added may be frothed.

As described above, the compound represented by the formula (1) can improve the recovery amount of a mineral, in particular, the recovery amount of a plurality of kinds of minerals. Therefore, since the flotation method of the present embodiment using the compound represented by the formula (1) can float a larger amount of minerals on the liquid surface of the ore slurry compared to conventional collectors, the amount of minerals recovered from the ore slurry can be increased.

Furthermore, in the flotation method of the present embodiment, the ore slurry is preferably a slurry of ore containing one or more kinds of minerals each including one or more kinds of metals selected from the group consisting of Cu, Au, Zn, Pb, Pt, Pd, Rh, Ni, and Co, and more preferably a slurry of ore containing one or more kinds of minerals each including one or more kinds of metals selected from the group consisting of Cu, Au, Zn, and Pb.

As described above, the compound represented by the formula (1) can particularly improve the recovery amount of minerals including the above metals. Therefore, by flotation of one or more kinds of minerals each including the one or more kinds of metals in the flotation method of the present embodiment, the recovery amount of the one or more kinds of minerals can be increased.

In the flotation method, the addition amount of the compound represented by the formula (1) is preferably 0.1 g or more and 1000 g or less, more preferably 10 g or more and 300 g or less, and still more preferably 50 g or more and 300 g or less, per 1000 kg of the ore. When the addition amount of the compound represented by the formula (1) is 0.1 g or more, the recovery amount of the target mineral can be sufficiently increased. When the addition amount of the compound represented by the formula (1) is 1000 g or less, the cost of the used compound represented by the formula (1) can be reduced.

In the flotation method, the pH of the ore slurry is preferably 6 or more and 12 or less, and is more preferably 9 or more and 12 or less. When the pH of the ore slurry is within the above range, the recovery amount of the mineral can be further increased.

Next, a compound of an embodiment of the present disclosure will be described.

The compound of the present embodiment is a compound represented by the above formula (3-1-1).

Among the compounds represented by the above formula (1), with respect to the compound represented by the formula (3-1-1), two sulfur atoms present in adjacent positions strongly bind to a mineral. Therefore, the compound represented by the formula (3-1-1) is suitably used in a collector.

The compound represented by the formula (3-1-1) can be synthesized by a reaction represented by the following reaction formula.

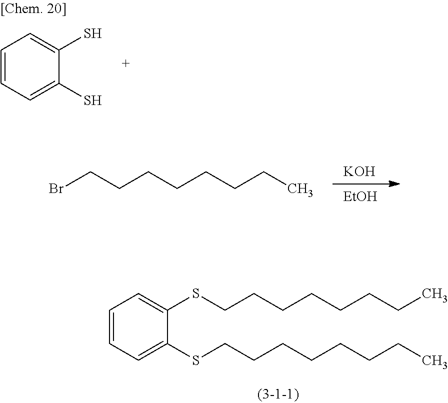

First, under a nitrogen gas flow, 1,2-benzenedithiol, 1-bromooctane, and potassium hydroxide are heated and refluxed while stirring in ethanol. Subsequently, the obtained sample is washed, dehydrated, and dried. Thus, the compound represented by the formula (3-1-1) can be obtained.

According to the embodiment described above, the recovery amount of the target mineral can be increased by using the collector including the compound represented by the formula (1). Therefore, in flotation, the recovery amount of desired mineral can be increased.

### EXAMPLES

Next, examples and comparative examples will be described, but the present disclosure is not limited to these examples.

After preparing the compounds shown above, each example was carried out using a collector including each compound.

### (Synthesis of Formula (2-2-1))

Under a nitrogen gas flow, 2-aminobenzenethiol (0.5 g), potassium hydroxide (0.5 g), and 1-bromooctane (0.77 g) were heated and refluxed at 75° C. for 1 hour while stirring in acetone (30 mL). Subsequently, the obtained sample was washed, dehydrated, and vacuum-dried. As a result of analyzing the obtained sample by NMR, the compound represented by the formula (2-2-1) could be obtained in a yield of 80.1%. This compound was brownish-red and oily.

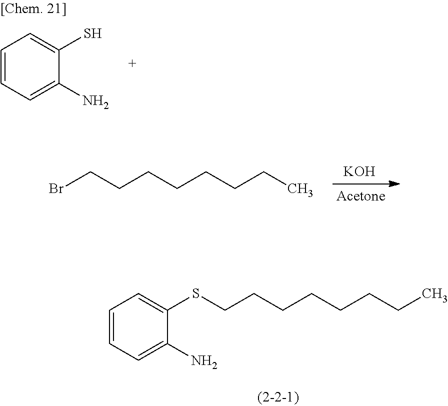

### (Synthesis of Formula (2-2-2))

Under a nitrogen gas flow, 3-aminobenzenethiol (0.5 g), potassium hydroxide (0.5 g), and 1-bromooctane (0.77 g) were heated and refluxed at 75° C. for 1 hour while stirring in acetone (30 mL). Subsequently, the obtained sample was washed, dehydrated, and vacuum-dried. As a result of analyzing the obtained sample by NMR, the compound represented by the formula (2-2-2) could be obtained in a yield of 82.5%. The compound was reddish brown and oily.

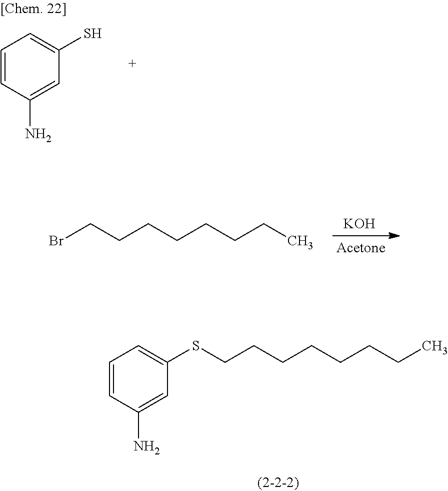

### (Synthesis of Formula (2-2-3))

Under a nitrogen gas flow, 4-aminobenzenethiol (0.5 g), potassium hydroxide (0.5 g), and 1-bromooctane (0.77 g) were heated and refluxed at 75° C. for 1 hour while stirring in acetone (30 mL). Subsequently, the obtained sample was washed, dehydrated, and vacuum-dried. As a result of analyzing the obtained sample by NMR, the compound represented by the formula (2-2-3) could be obtained in a yield of 85.0%. This compound was brownish-black and oily.

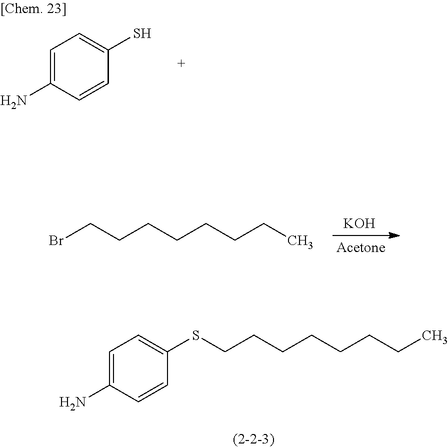

### (Synthesis of Formula (3-1-1))

Under a nitrogen gas flow, 1,2-benzenedithiol (0.5 g), potassium hydroxide (1.57 g), and 1-bromooctane (3.35 g) were heated and refluxed at 100° C. for 1 hour while stirring in ethanol (24 mL). Subsequently, the obtained sample was washed, dehydrated, and vacuum-dried. As a result of analyzing the obtained sample by NMR, the compound represented by the formula (3-1-1) could be obtained in a yield of 92.3%. This compound was yellow and oily.

¹H-NMR (CDCl₃, 500 MHz) of formula (3-1-1): δ0.88 (t, 6H), δ1.28 (br, 16H), δ1.44 (tt, 4H), δ1.67 (tt, 4H), δ2.90 (t, 4H), δ7.12 (dd, 2H), and δ7.26 (dd, 2H)

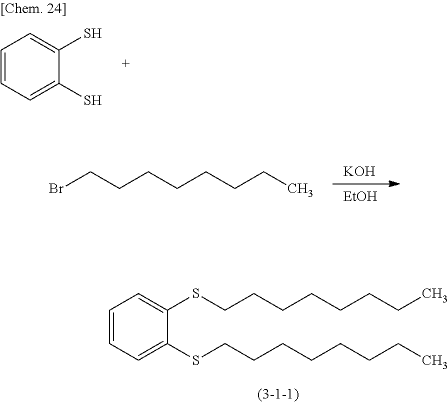

### (Synthesis of Formula (3-1-2))

Under a nitrogen gas flow, 1,3-benzenedithiol (0.5 g), potassium hydroxide (1.57 g), and 1-bromooctane (3.35 g) were heated and refluxed at 100° C. for 1 hour while stirring in ethanol (24 mL). Subsequently, the obtained sample was washed, dehydrated, and vacuum-dried. As a result of analyzing the obtained sample by NMR, the compound represented by the formula (3-1-2) could be obtained in a yield of 81.4%. This compound was yellow and oily.

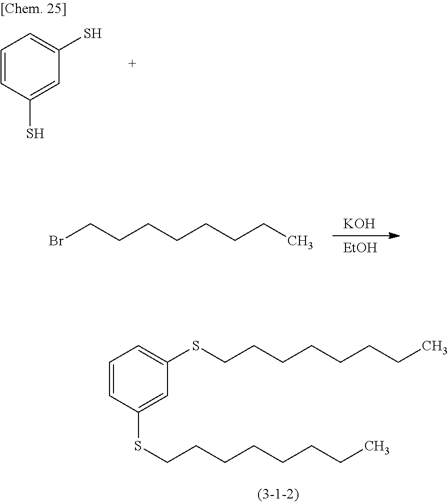

### (Synthesis of Formula (3-1-3))

Under a nitrogen gas flow, 1,4-benzenedithiol (0.5 g), potassium hydroxide (1.57 g), and 1-bromooctane (3.35 g) were heated and refluxed at 100° C. for 1 hour while stirring in ethanol (24 mL). Subsequently, the obtained sample was washed, dehydrated, and vacuum-dried. As a result of analyzing the obtained sample by NMR, the compound represented by the formula (3-1-3) could be obtained in a yield of 75.2%. This compound was a pale yellow crystal.

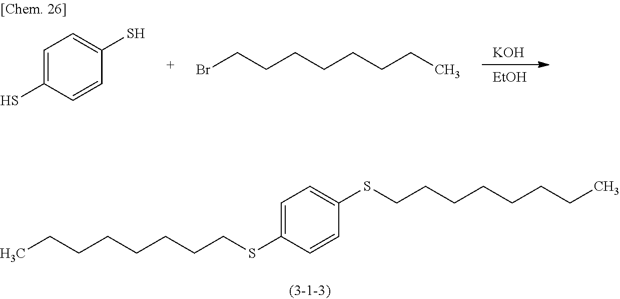

### Examples 1-1 to 1-6 and Comparative Example 1-1

As shown in Table 1, as each of Examples 1-1 to 1-6, flotation of chalcopyrite was performed using the compound represented by each of the formulas (2-2-1) to (2-2-3) and the formulas (3-1-1) to (3-1-3). As Comparative Example 1-1, flotation of the chalcopyrite was performed using PAX (Cytec Industries Inc., potassium amyl xanthate).

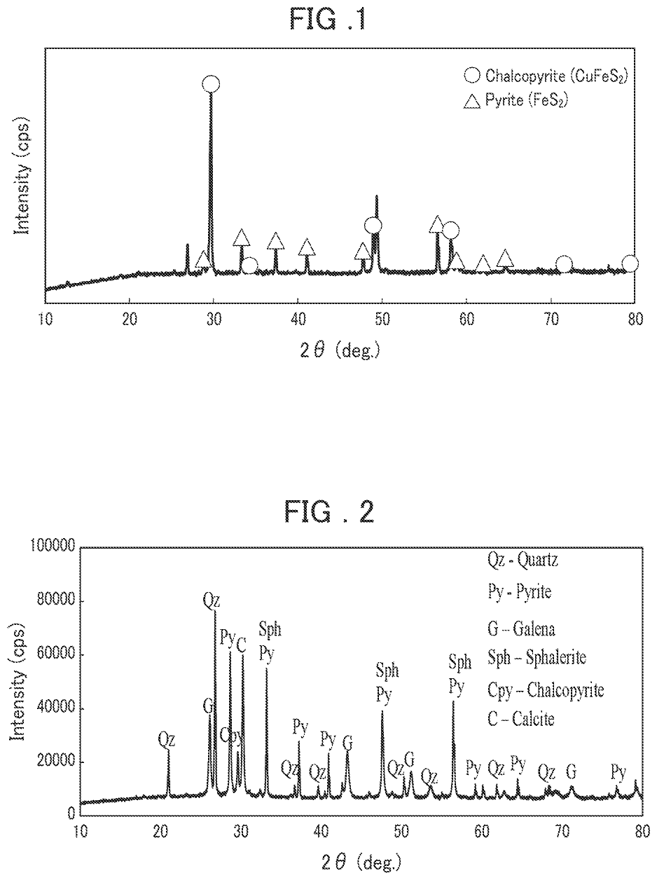

The flotation was performed according to the following procedure. First, a slurry of pulverized chalcopyrite with the X-ray diffraction pattern shown in FIG. 1, which was pulverized to a particle size of 75 um or less, was adjusted to pH 9, and then 50 g of the compound represented by each formula or PAX and 100 g of a frother (methylisobutylcarbinol (MIBC)) were added per 1000 kg of the chalcopyrite. The Cu grade and the Fe grade of the used chalcopyrite were respectively 27.5% and 40.9%. The amount of the pulverized chalcopyrite in the slurry was 25 g. Note that the compound represented by the formula (3-1-3) was dissolved in kerosene, and then added to the slurry. The compounds other than the compound represented by the formula (3-1-3) were directly added to the slurry. Then, flotation was performed for 10 minutes to obtain floating ore and tailings. The copper grades of the obtained floating ore and the tailings were analyzed, and the recovery rate of Cu was calculated based on the following equation. The floating ore is ore that floats during flotation, and the tailings are ore that does not float during flotation. The Cu grade shown in Table 1 is the copper grade of the floating ore.

Recovery rate (%) of Cu=weight of floating ore×copper grade of floating ore/(weight of floating ore×copper grade of floating ore+weight of tailings×copper grade of tailings)

TABLE 1

| | | | Amount of compound | | |
|---|---|---|---|---|---|
| | | | relative to mineral | Cu recovery rate | Cu grade |
| | Compound | Mineral | (g/t) | (%) | (%) |
| Example 1-1 | Formula (2-2-1) | Chalcopyrite | 50 | 85.4 | 37.9 |
| Example 1-2 | Formula (2-2-2) | Chalcopyrite | 50 | 85.3 | 35.9 |
| Example 1-3 | Formula (2-2-3) | Chalcopyrite | 50 | 62.0 | 40.2 |
| Example 1-4 | Formula (3-1-1) | Chalcopyrite | 50 | 39.4 | 37.3 |
| Example 1-5 | Formula (3-1-2) | Chalcopyrite | 50 | 20.4 | 29.4 |
| Example 1-6 | Formula (3-1-3) | Chalcopyrite | 50 | 28.8 | 42.0 |
| Comparative | PAX | Chalcopyrite | 50 | 7.1 | 38.6 |
| Example 1-1 | | | | | |

As shown in Table 1, in Examples 1-1 to 1-6, the recovery rate of Cu increased compared to Comparative Example 1-1.

### Examples 2-1 to 2-4 and Comparative Example 2-1

Flotation was performed by the same manner as in Example 1-1 except that each compound was used and 100 g of each compound was added per 1000 kg of the chalcopyrite as shown in Table 2.

TABLE 2

| | | | Amount of compound | | |
|---|---|---|---|---|---|
| | | | relative to mineral | Cu recovery rate | Cu grade |
| | Compound | Mineral | (g/t) | (%) | (%) |
| Example 2-1 | Formula (2-2-1) | Chalcopyrite | 100 | 92.5 | 37.2 |
| Example 2-2 | Formula (2-2-2) | Chalcopyrite | 100 | 92.1 | 35.1 |
| Example 2-3 | Formula (2-2-3) | Chalcopyrite | 100 | 85.0 | 36.5 |
| Example 2-4 | Formula (3-1-1) | Chalcopyrite | 100 | 63.3 | 39.7 |
| Comparative | PAX | Chalcopyrite | 100 | 60.1 | 39.8 |
| Example 2-1 | | | | | |

As shown in Table 2, in Examples 2-1 to 2-4, the recovery rate of Cu increased compared to Comparative Example 2-1.

### Examples 3-1 to 3-3 and Comparative Example 3-1

Flotation was performed by the same manner as in Example 1-1 except that each compound was used and 300 g of each compound was added per 1000 kg of the chalcopyrite as shown in Table 3.

TABLE 3

| | | | Amount of compound | | |
|---|---|---|---|---|---|
| | | | relative to mineral | Cu recovery rate | Cu grade |
| | Compound | Mineral | (g/t) | (%) | (%) |
| Example 3-1 | Formula (2-2-1) | Chalcopyrite | 300 | 95.6 | 36.7 |
| Example 3-2 | Formula (2-2-2) | Chalcopyrite | 300 | 94.4 | 33.3 |
| Example 3-3 | Formula (2-2-3) | Chalcopyrite | 300 | 73.7 | 36.4 |
| Comparative | PAX | Chalcopyrite | 300 | 66.5 | 37.3 |
| Example 3-1 | | | | | |

As shown in Table 3, in Examples 3-1 to 3-3, the recovery rate of Cu increased compared to Comparative Example 3-1.

### Example 4-1 and Comparative Example 4-1

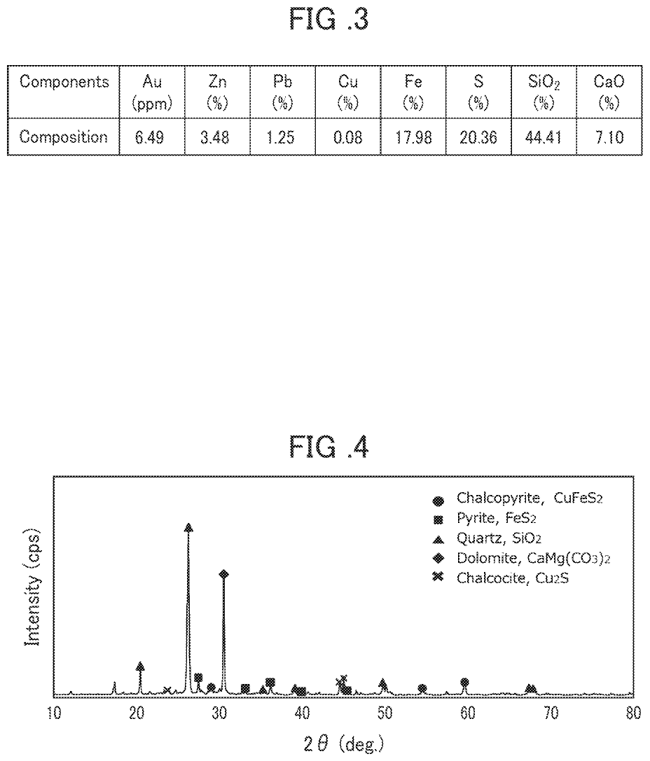

As shown in Table 4, as each of Example 4-1 and Comparative Example 4-1, flotation of the ore with the X-ray diffraction pattern shown in FIG. 2 was performed using the compound represented by the formula (2-2-1) or PAX. As a result of analyzing this ore by MP-AES and XRF, it was found that the ore contains various metals shown in FIG. 3.

The flotation was performed according to the following procedure. First, a slurry of pulverized ore, which was pulverized to a particle size of 75 μm or less, was adjusted to pH 7 to 8, and then 100 g of the compound represented by the formula (2-2-1) or PAX and 200 g of a frother (methylisobutylcarbinol (MIBC)) were added per 1000 kg of the ore. The amount of the pulverized ore in the slurry was 75 g, and the pulp concentration was 30%. Then, flotation was performed for 15 minutes to calculate the recovery rates of Au, Zn, and Pb.

TABLE 4

| | | | Amount of | | | |
|---|---|---|---|---|---|---|
| | | | compound | Au | Zn | Pb |
| | | | relative | recovery | recovery | recovery |
| | | | to ore | rate | rate | rate |
| | Compound | Ore | (g/t) | (%) | (%) | (%) |
| Example 4-1 | Formula (2-2-1) | Lead-zinc gold ore | 100 | 50.0 | 12.4 | 44.0 |
| Comparative | PAX | Lead-zinc gold ore | 100 | 30.5 | 9.4 | 40.7 |
| Example 4-1 | | | | | | |

As shown in Table 4, in Example 4-1, all of the recovery rate of Au, the recovery rate of Zn, and the recovery rate of Pb increased compared to Comparative Example 4-1.

### Examples 5-1 to 5-8 and Comparative Examples 5-1 and 5-2

As shown in Table 5, as each of Examples 5-1 to 5-8, flotation of copper sulfide ore was performed using the compound represented by each of formulas (2-2-1) to (2-2-3) and (3-1-1). Furthermore, as each of Comparative Examples 5-1 and 5-2, flotation of the copper sulfide ore was performed using PAX.

The flotation was performed according to the following procedure. First, a slurry of pulverized copper sulfide ore with the X-ray diffraction pattern shown in FIG. 4, which was pulverized to a particle size of 75 μm or less, was adjusted to pH 9 or 12, and then 100 g of the compound represented by each formula or PAX and 100 g of a frother (methylisobutylcarbinol (MIBC)) were added per 1000 kg of the copper sulfide ore. The Cu grade and the Fe grade of the used copper sulfide ore were respectively 3.0% and 4.2%. The amount of the pulverized ore in the slurry was 25 g. Note that PAX was dissolved in water, and then added to the slurry, and the compounds other than PAX were directly added to the slurries. Then, flotation was performed for 10 minutes to obtain the floating ore and the tailings.

TABLE 5

| | | | Cu recovery rate | Cu grade |
|---|---|---|---|---|
| | Compound | pH | (%) | (%) |
| Example 5-1 | Formula (2-2-1) | 9 | 49.4 | 13.3 |
| Example 5-2 | Formula (2-2-2) | 9 | 46.0 | 13.1 |
| Example 5-3 | Formula (2-2-3) | 9 | 41.1 | 12.7 |
| Example 5-4 | Formula (3-1-1) | 9 | 36.2 | 12.4 |
| Example 5-5 | Formula (2-2-1) | 12 | 68.9 | 15.1 |
| Example 5-6 | Formula (2-2-2) | 12 | 62.0 | 14.5 |
| Example 5-7 | Formula (2-2-3) | 12 | 55.7 | 14.2 |
| Example 5-8 | Formula (3-1-1) | 12 | 49.3 | 13.5 |
| Comparative | PAX | 9 | 30.7 | 12.0 |
| Example 5-1 | | | | |
| Comparative | PAX | 12 | 43.0 | 12.5 |
| Example 5-2 | | | | |

As shown in Table 5, in each of Examples 5-1 to 5-4, the recovery rate of Cu increased and the Cu grade improved compared to Comparative Example 5-1, and in each of Examples 5-5 to 5-8, the recovery rate of Cu increased and the Cu grade improved compared to Comparative Example 5-2.

1. A collector comprising a compound represented by the following formula (1): 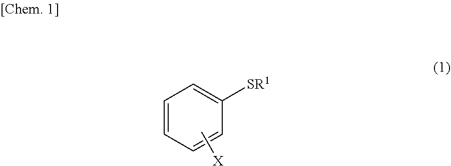
wherein R¹ represents an alkyl group having 1 or more and 18 or less carbon atoms, and X represents NR²R³, SR⁴, or OR⁵, and
R²⁻³ each represent hydrogen or an alkyl group having 1 or more and 18 or less carbon atoms, and R⁴⁻⁵ each represent an alkyl group having 1 or more and 18 or less carbon atoms.

2. The collector according to claim 1, wherein R¹ represents an alkyl group having 5 or more and 10 or less carbon atoms.

3. The collector according to claim 1, wherein X represents NR²R³ or SR⁴.

4. The collector according to claim 1, wherein R² and R³ are each hydrogen.

5. The collector according to claim 1, wherein R⁴ represents an alkyl group having 5 or more and 10 or less carbon atoms.

6. The collector according to claim 1, wherein R⁵ represents an alkyl group having 5 or more and 10 or less carbon atoms.

7. The collector according to claim 1, wherein the collector is a collector for flotation of one or more kinds of minerals each including one or more kinds of metals selected from the group consisting of Cu, Au, Zn, Pb, Pt, Pd, Rh, Ni, and Co.

8. A flotation method comprising: adding a compound represented by the following formula (1) and a frother to an ore slurry to float one or more kinds of minerals in the ore slurry and recovering the one or more kinds of minerals: 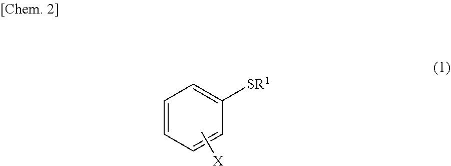
wherein R¹ represents an alkyl group having 1 or more and 18 or less carbon atoms, and X represents NR²R³, SR⁴, or OR⁵, and
R²⁻³ each represent hydrogen or an alkyl group having 1 or more and 18 or less carbon atoms, and R⁴⁻⁵ each represent an alkyl group having 1 or more and 18 or less carbon atoms.

9. The flotation method according to claim 8, wherein the ore slurry is a slurry of ore containing one or more kinds of minerals each including one or more kinds of metals selected from the group consisting of Cu, Au, Zn, Pb, Pt, Pd, Rh, Ni, and Co.

10. The flotation method according to claim 8, wherein an addition amount of the compound represented by the formula (1) is 0.1 g or more and 1000 g or less per 1000 kg of ore.

11. The flotation method according to claim 8, wherein the ore slurry has a pH of 6 or more and 12 or less.

12. A compound represented by the following formula (3-1-1): 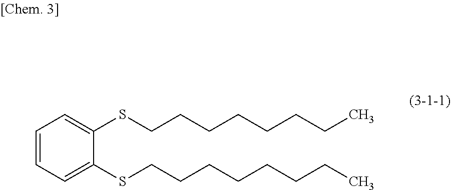 [SMILES: CCCCCCCCSc1ccccc1SCCCCCCCC]
